(19) United States
(12) Patent Application Publication
(10) Pub. No.: US 2008/0221218 A1
(43) Pub. Date: Sep. 11, 2008

(54) Receptor

(75) Inventors: Hubertus Franciscus Martinus Nelissen, York (GB); David Kelham Smith, York (GB)

Correspondence Address: TROUTMAN SANDERS LLP, 600 PEACHTREE STREET , NE, ATLANTA, GA, 30308, US

(73) Assignee: The University of York, York (GB)

(21) Appl. No.: 12/064,032

(22) PCT Filed: Aug. 15, 2006
(86) PCT No.: PCT/GB2006/003036

§ 371 Date: Mar. 20, 2008

(30) Foreign Application Priority Data
Aug. 18, 2005 (GB) 0516946.1

(51) Int. Cl. A61K 31/16; C07C 237/24; A61P 13/00; B01D 39/04; C02F 1/42; C07C 237/16

(52) U.S. Cl. 514/627; 564/152; 564/160; 210/683; 210/502.1

(57) ABSTRACT

The present invention relates to synthetic receptors, products and methods which are useful in relation to the binding of phosphates for example in water purification and renal dialysis systems.

### FIELD OF THE INVENTION

The present invention relates to synthetic receptors, products and methods which are useful in relation to the binding of phosphates for example in water purification and renal dialysis systems.

### BACKGROUND TO THE INVENTION

Phosphates are ubiquitous in nature; they make up the backbone of DNA and RNA, and provide a source of chemical energy in the form of ATP. Phosphate anions also play an important role in causing eutrophication of surface water and, medically, high phosphate levels in end stage renal failure patients are related to increased morbidity and mortality. For these reasons phosphates have been an interesting target for synthetic receptors.

A class of receptors that has been studied in this context is polyamines, which mimic one of nature's very own phosphate binders, spermine. These synthetic receptors bind phosphate using a combination of electrostatic interactions and hydrogen bonds between the protonated amines and the H₂PO₄⁻ and HPO₄²⁻ anions, the two main species at neutral pH.

One inherent challenge encountered in the binding of phosphate anions is to establish the binding in water. As a highly competitive solvent, water drastically attenuates the main driving forces for binding, i.e. electrostatic interactions and hydrogen bonding.

A known strategy to overcome the weak affinity of polyamine receptors for phosphate in water is to form a rigid macrocyclic ring. Organising the protonatable amines and hydrogen bond donors/acceptors in a ring structure reduces the conformational flexibility of the receptor and has a pre-organising effect resulting in a higher affinity mainly due to entropic reasons. This phenomenon is known as the macrocyclic effect. Although having increased binding strength for phosphate in comparison with open chain receptors, macrocycles are synthetically challenging because their synthesis usually requires multi-step reactions and dilute reaction conditions to promote the final macrocyclisation over competing polymerisation reactions.

Kubik et al. (Angew. Chem. Int. Ed. 2001, 40 No. 14: 2648-2651) investigated the binding of anions, including phosphate, in a 80% water/methanol mixture using cyclic peptides containing only amide bonds as interaction sites. In general amide based receptors only operate well in organic media and are not very well suited for partially protonated anions such as phosphate.

D. A. Nation et al. (Inorg. Chem. 35:4597-4603 (1996)) investigated a series of polyamine macrocycles that can only interact via electrostatic interactions. These are able to bind phosphate in water with association constants of 2.5-3.9, depending on the degree of protonation of the receptor. Although appreciable, the binding is only significant up to pH 6, severely limiting the potential applications.

Hossain et al. (Inorg. Chem. 42:1397-1399 (2003) describe marcocyles obtained by combining amides with quaternised amines. Binding of these macrocyles to phosphate was tested in organic solvents such as dimethylsulfoxide but not in water. There is a requirement for improved structures, for example synthetically more easily accessible structures, which can be used to bind phosphates with high affinity in water.

### STATEMENTS OF THE INVENTION

According to a first aspect of the present invention there is provided a ligand binding member comprising
i) at least two oligoamines arranged for binding to a ligand; and
ii) an organic template covalently linked to the at least two oligoamines such that movement of the oligoamines is conformationally restricted wherein the oligoamines are the same or different and are independently straight chained, cyclic or branched.

As used herein, the term “ligand binding member” includes any entity that can form a complex with a ligand. Typically, the ligand binding member is a receptor and the ligand is a substrate of the receptor.

As used herein a “ligand” may include a molecule or ion that is capable of binding to the ligand binding member.

The ligand may be a phosphate molecule, phosphate containing molecule or phosphate ion. Phosphate molecules may include inorganic phosphates or organic phosphates such as described herein.

The terms “phosphate containing molecule” and “phosphorylated target” may be used interchangeably to mean any molecule containing or presenting one or more phosphate molecules. The molecule containing the phosphate, or the target, may be a biological molecule (for example a protein, lipid, carbohydrate or nucleic acid (DNA or RNA)) or any organic molecule.

As used herein an “oligoamine” includes a plurality of joined amine groups. A straight chained oligoamine generally relates to a straight chain carbon backbone with a plurality of amine substituents. A branched or cyclic oligoamine generally relates to a respective branched or cyclic carbon chain backbone with a plurality of amine substituents. The carbon chain backbone may comprise up to 20 carbon atoms, such as 1 to 10 or 1 to 5 carbon atoms, for example 1, 2, 3, 4 or 5 carbon atoms.

As used herein an “organic template” includes any organic molecule which functions as a scaffold for the oligoamines.

As used herein the expression “conformationally restricted” relates to the restriction on movement, for example rotation, of the oligoamines about a single chemical bond. The restriction on the conformation of the oligoamines results in a defined relationship of one oligoamine to another such that binding of each one of them to the ligand is enhanced, for example, the conformational restriction gives rise to a ligand binding pocket being provided by the oligoamines.

The ligand binding member may consist of two oligoamines which may be the same or different. Preferably, the oligoamines are the same.

The relative positions of the at least two oligoamines in the ligand binding member may be cis (or Z) or trans (or E).

The at least two oligoamines of the ligand binding member may independently be acyclic oligoamines. The oligoamines may independently comprise an optionally substituted terminal amine group, for example, a terminal NH₂ group.

Preferably the organic template of the ligand binding member according to the invention is an unsaturated hydrocarbon, a cyclic aliphatic hydrocarbon or a cyclic aromatic hydrocarbon. Such templates serve to restrict the relative conformation of the oligoamines attached to the template. The unsaturated hydrocarbon may be alkenyl or alkynyl. Preferably the unsaturated hydrocarbon is alkenyl. The cyclic aromatic hydrocarbon may be a monocyclic or polycyclic aromatic hydrocarbon. A monocyclic aromatic hydrocarbon may include benzene or pyran. Polycyclic aromatic hydrocarbons may comprise two, three or four hydrocarbon rings. The polycyclic aromatic hydrocarbon may be selected from the group consisting of naphthalene, indene, pentalene, azulene, heptalene, biphenylene, indacene, fluorene, phenalene, phenanthrene, anthracene, pyrene, chrysene, naphthacene, acephananthrylene, aceanthrylene, triphenylene or fluoroanthene. The cyclic aliphatic hydrocarbon of the ligand binding member of the invention may be a monocyclic or polycyclic aliphatic hydrocarbon. Monocyclic aliphatic hydrocarbons may comprise between 3 and 10 carbon atoms. Preferably the monocyclic aliphatic hydrocarbon is cyclohexyl. Polycyclic aliphatic hydrocarbon may comprise two, three or four hydrocarbon rings. The polycyclic aliphatic hydrocarbon may be a bicyclic ring system.

As used herein “alkyl” may have up to 20, for example up to 12 carbon atoms and is linear or branched one or more times; preferred is lower alkyl, especially preferred is C₁-C₄-alkyl, in particular methyl, ethyl or i-propyl or t-butyl, where alkyl may be substituted by one or more substituents.

The term “alkenyl” as used herein refers to a straight or branched chain alkyl moiety having from two to six carbon atoms and having, in addition, at least one double bond, of either E or Z stereochemistry where applicable. This term refers to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

The term “alkynyl” as used herein refers to a straight or branched chain alkyl moiety having from two to six carbon atoms and having, in addition, at least one triple bond. This term refers to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

The term “amine” group is a nitrogen containing moiety, usually with at least two of its substitution sites occupied by hydrogen. An amino group having less than two substitution sites occupied by hydrogen is a mono- or di-substituted amino moiety.

An alkyl or amine group may be unsubstituted or substituted by a hydrocarbyl moiety, the hydrocarbyl moiety being, for example, selected from C1-4 alkyl, especially C₁, C₂, C₃ or C₄ alkyl, cycloalkyl, especially cyclohexyl, alkyl-carboxy, carboxy, alkanoyl, especially acetyl, a carbocyclic group, for example cyclohexyl or phenyl, a heterocyclic group; where the hydrocarbyl moiety is unsubstituted or substituted by, for example alkyl (C₁, C₂, C₃, C₄, C₅, C₆ or C₇), halogen, OH, esterified carboxy, etherified hydroxy, C1-6 alkoxy, NH₂, SH, S-alkyl, SO-alkyl, SO₂-alkyl, NH-alkyl, N-dialkyl, carboxyl, CF₃, wherein alkyl may be unsubstituted or substituted branched, unbranched or cyclic C₁₋₆, interrupted 0-3 times by O, S, N.

In the ligand binding member of the invention the template may be a heterocyclic hydrocarbon, for example a monocyclic heterocycle or a polycyclic heterocycle.

Examples of cyclic aliphatic or aromatic hydrocarbon groups, including heterocyles of these, include but are not limited to cyclohexyl, phenyl, acridine, benzimidazole, benzofuran, benzothiophene, benzoxazole, benzothiazole, carbazole, cinnoline, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isooxazole, isothiazole, morpholine, napthyridine, norbornene, oxazole, oxadiazole, oxathiazole, oxathiazolidine, oxazine, oxadiazine, phenazine, phenothiazine, phenoxazine, phthalazine, piperazine, piperidine, pteridine, purine, putrescine, pyran, pyrazine, pyrazole, pyrazoline, pyrazolidine, pyridazine, pyridine, pyrimidine, pyrrolidine, pyrrole, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, trithiane, tropine. Preferably the aromatic hydrocarbon is phenyl.

The ligand binding member may comprise a template which is a sugar, for example a pentose or hexose sugar, or glycoside.

In the ligand binding member the at least two oligoamines may be made up of primary, secondary or tertiary amines, quaternary ammonium salts or a combination thereof. Preferably the at least two oligoamines comprise a primary amine group and a secondary amine group.

In a preferred aspect of the invention the oligoamines contain between 2 and 4 protonatable amines, for example 3 or 4 protonatable amines. One or more of the oligoamines may comprise one or more amide groups.

A ligand binding member as claimed in any preceding claim wherein the amine groups of the oligoamines are separated by at least two atoms, for example 3 or 4 atoms, whereby the atoms can be carbon, oxygen or sulphur. Separating the protonatable amine groups ensures a sufficient degree of protonation, and thereby enhanced electrostatic interactions, with the bound ligand. In this way, ligand binding is enhanced.

The at least two atoms separating the amine groups may be hydrocarbon groups such as C1-6 alkyl, for example methyl or ethyl.

Thus in a preferred embodiment of the invention, the oligoamine is of formula (I)

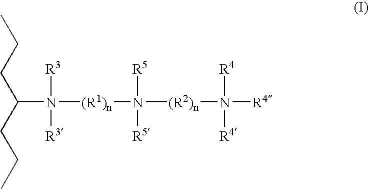

wherein R¹ is independently selected from O, S, C1-4 alkyl (e.g methylene) or a combination thereof; R² is independently selected from O, S, C1-4 alkyl (e.g methylene) or a combination thereof; R³ and R^{3′} are independently H or C1-4 alkyl (e.g methyl); R⁴, R^{4′} and R^{4″} are independently H or C1-4 alkyl (e.g methyl); R⁵ and R^{5′} are independently H or C1-4 alkyl (e.g methyl) wherein R⁴ may be absent; and n is an integer between 1 and 5 (e.g 1, 2 or 3).

In one embodiment of the invention, one or more of R⁴, R^{4′} and R^{4″} (typically C1-4 alkyl) from the at least two oligoamines of the ligand binding member may be joined together to form a cyclic hydrocarbon. Alternatively, one or more of R⁴, R^{4′} and R^{4″} (typically methyl or ethyl) from the at least two oligoamines of the ligand binding member may be joined via a cyclic hydrocarbon, for example a phenyl group. Thus the invention provides ligand binding members that are cyclic.

The covalent linkage (indirect or direct attachment) of each of the at least two oligoamines to the conformationally restricted scaffold may be via amide bonds. The present inventors have surprisingly found that the presence of amide groups in the ligand binding member has pronounced effects on its structure in aqueous solution. The amide groups provide an additional conformational restraint to the ligand binding member.

Thus in a preferred embodiment of the invention, the ligand binding member is of formula (II)

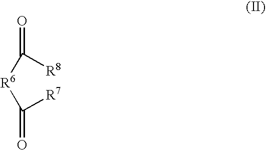

wherein R⁶ is an organic template as defined; and each of R⁷ and R⁸ is independently an oligoamine as defined herein.

In a preferred aspect of the invention the ligand binding member is a receptor. The receptor may be an acyclic or cyclic receptor.

Preferably the receptor is an acyclic receptor. As used herein, the term “acyclic” can be used interchangeably with open-chain or linear.

In a preferred embodiment of the invention the ligand binding member is an acyclic compound as shown in 1, or 2 below

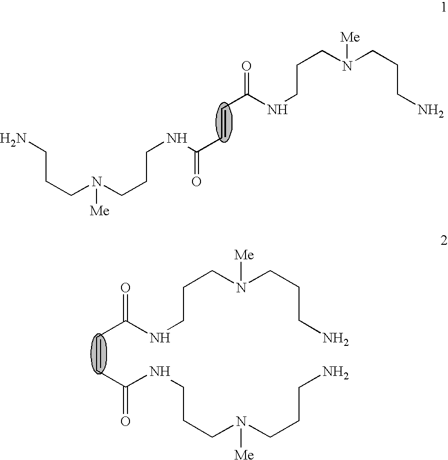

In a further embodiment of the invention the ligand binding member of is a cyclic compound of structure 3:

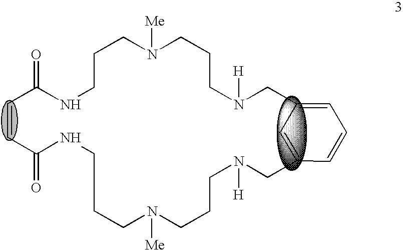

Preferred embodiments of the invention include the following ligand binding members:

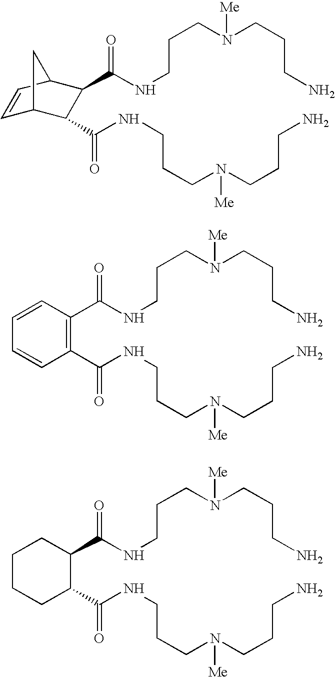

In a further preferred aspect of the invention the receptor is synthetic.

In a preferred aspect of the invention the ligand is a phosphate molecule or a molecule that contains at least one phosphate group. Phosphate molecules may include inorganic phosphates, i.e. phosphates containing no carbon, such as ortho-phosphate (PO₄³⁻) and pyro-phosphate (P₂O₇⁴⁻). Organic phosphate molecules may include monophosphates, diphosphates, triphosphates or polyphosphates. Examples of phosphate containing organic molecules include adenosine di-phosphate and adenosine tri-phosphate. The ligand may be attached to a biological molecule, for example, the ligand may be attached to a protein, lipid, carbohydrate or nucleic acid.

The ligand binding member of the invention may, as a result of binding to a phosphate containing molecule, be a phosphatase inhibitor.

In a preferred aspect of the invention the ligand binding member is for use as a medicament. Thus the ligand binding member may be provided as a pharmaceutical composition in conjunction with a pharmaceutically acceptable carrier or diluent.

The invention includes the described ligand binding members in all their forms, including for example their isomers, prodrugs and pharmaceutically acceptable salts.

The ligand binding member may be adapted for oral administration, for example, in the form of a tablet or capsule.

The ligand binding member of the invention may be immobilised on a support. Preferably the support is a solid support. Preferably the ligand binding member is covalently attached to the solid support. The solid support may comprise a polymer, for example, including but not limited to vinyl type polymers, acrylic polymers, allyl polymers (e.g polyallylamine and polyallylalcohol) or polyethyleneoxides. Polymers may also include agarose, sepharose, dextran, cellulose, xylan, lignin, nylon, DNA, RNA, polyester, polyamide, polycarbonate, and derivatives thereof. Polymers may be homopolymers or any combination or copolymer of the above. Specific polymers may include polystyrene, polyethylene, polypropylene, polymethylmethacrylate, polytetrafluoroethylene and halogenated derivatives of any of the above, polyvinyl alcohol, polyvinyl chloride, polyphosphate, polyethylene glycol, polybutadiene, polypeptide, polyacrylamide, polyacrylic acid, polyacrylates, polymethacrylates, polyacrylamide, poly(N-alkyl)acrylamides, polyhema, or any combination or copolymer of the above.

The support may be in the form of beads for example polymer beads, glass beads, or silica beads. Alternatively, the solid support may comprise a microtitre plate or a glass slide, or may be composed of a gel, fibres, filaments, membrane, dendrites, resins, micelles, nanoparticles or phase interfaces. The support may form part of a column, filtration device, mesh or gel.

In a second aspect of the invention there is provided a filter comprising a ligand binding member according to the first aspect of the invention.

In a further aspect of the invention there is provided a water purification system comprising a filter according to the second aspect of the invention.

In a yet further aspect of the invention there is provided a renal dialysis system comprising a filter according to the second aspect of the invention.

A further aspect of the invention provides the use of a ligand binding member according to the first aspect of the invention in the removal of phosphate molecules, phosphate ions or phosphate containing molecules, such as defined herein, from a liquid. The liquid may, for example, be water, effluent, urine or blood.

In a further aspect of the invention there is provided the use of a ligand binding member according to the first aspect of the invention in the selective screening of phosphate molecules or phosphate containing molecules.

Preferably the ligand binding member is a cyclic (e.g or macrocyclic) structure, for example a cyclic receptor.

Both acyclic and cyclic (such as macrocyclic) receptors according to the invention have been shown to bind inorganic phosphate molecules more effectively than phosphate containing organic molecules. Thus the invention provides the use of an acyclic or cyclic ligand binding member according to the invention in the selective screening, or removal (e.g from a liquid), of an inorganic phosphate molecule (for example free phosphate in solution). Thus the ligand binding members according to the invention may facilitate the separation of inorganic phosphates from other phosphates such as phosphate containing organic molecules.

The invention further provides the use of an acyclic ligand binding member according to the invention in the selective screening, or removal, of a phosphate containing molecule (for example phosphate containing organic molecules), for example from a liquid. Thus the ligand binding members according to the invention may facilitate the separation of phosphate containing molecules from other phosphates such as inorganic phosphates.

A yet further aspect of the invention provides a process for separating a phosphate molecule or ion, or phosphate containing molecule, from a liquid, the process comprising the steps of:
(i) contacting a ligand binding member according to the invention with a liquid at pH values between about 5 and 9, and
ii) optionally removing any phosphate molecule, ion or phosphate containing molecule which bound to the ligand binding member.

In a preferred process of the invention, step (i) is carried out at a pH between 6 and 8, even more preferably between pH 6.5 and 7.5

A further aspect of the invention provides the use of a ligand binding member according to the first aspect of the invention in the inhibition of phosphatase activity. Through attachment of a ligand binding member to a ligand, in this case phosphate containing molecule, the ligand binding member serves to block the activity of a phosphatase on the ligand.

The ligand binding member may be labelled to allow it to be detected and/or analysed or quantified. Thus the invention provides a ligand binding member according to the invention which comprises a detectable label. The label may comprise, for example, an enzyme, a fluorescent label or a radioisotope which is readily detectable. Such a labelled ligand binding member may be used to target phosphorylated epitopes, for example in cells.

A yet further aspect of the invention provides the use of a ligand binding member according to the first aspect of the invention in the manufacture of a medicament for the treatment or prevention of renal failure.

A further aspect of the invention provides a method of treating renal failure by prophylaxis or therapy, comprising administration to a subject of a therapeutically effective amount of a ligand binding member according to the first aspect of the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

FIG. 1: Part of the 270 MHz ¹H spectrum of compound 10 in [D6]DMSO at different temperatures;

FIG. 2: Part of the 270 MHz ¹H spectrum of compound 2 in D₂O at different temperatures showing both the asymmetric (•) and symmetric conformations;

FIG. 3: Fluorescence titration curves for the binding of 13 (5.0×10⁻⁶ M) to 2 (▪) and 3 () in a 0.1 M TRIS buffer of pH 7.0 at 20° C.

FIG. 4: Graphs of the fraction of phosphate bound to receptor (solid lines) and free in solution (dashed lines) by compounds 1 (A), 2 (B), 3 (C), and 14 (D).

### EXAMPLES

### Materials and Methods

### Potentiometric Titrations

Potentiometric measurements were conducted using a Metrohm 792SM Titrino automated burette fitted with a combined glass electrode and an in-house designed jacketed glass cell thermostated at 25° C. suitable for small sample volumes (5 mL). The cell was sealed under an inert atmosphere. All solutions were made up using ultrapure CO₂-free water from a Millipore Simplicity 185 system. The sample solutions contained 0.1 M KCl as a background electrolyte. The titrant base was made up from commercial (Riedel-de Haën) CO₂-free concentrates of KOH, diluted to the required concentration (0.020 M) with a 0.080 M KCl solution to avoid problems of different ionic strengths. The glass electrode was calibrated by titrating well-known amounts of HCl (made up from Riedel-de Haën concentrates) with the base and determining the equivalence point using Gran's method (G. Gran, Analyst, 1952, 77, 661-671) yielding the ionic product of water (pKw=13.78), which was used as a constant in the subsequent analyses.

Sample concentrations were typically 1.0-5.0×10⁻³ M. A known excess of HCl was added to ensure full protonation of the receptor. The degree of protonation of our receptors was calculated from the onset of titration curve. For the phosphate binding studies an equimolar mixture of KH₂PO₄ and receptor was used. A minimum of 250 points in the pH-range of 2.5-11.0 was taken for every titration, with at least 30 seconds between each addition to ensure equilibration. The computer program HYPERQUAD^{I} was used to determine the protonation and stability constants. For each system a minimum of three titrations were first treated as individual sets and then merged and fitted simultaneously to give the final constants.

### Fluorimetric Titrations

Titrations were performed on a Spex Fluoromax-II instrument at 25° C. in a 0.1 M TRIS buffer of pH 7.0, using an excitation wavelength of 324 nm. The fluorophore 4-methylumbelliferyl phosphate was purchased from Sigma and its concentration was kept constant at 5.0×10⁻⁶ M. The data was fitted to a 1:1 binding model with a non-linear least-squares algorithm.

### Receptor Synthesis and Binding Experiments

Two examples of receptors are shown in Scheme 1 (Compounds 1 and 2). The conformationally restricted scaffolds present in the structures are indicated by the grey areas. Amides are used to covalently link the oligoamines to the scaffold. The amide bonds also provide additional conformational restrictions due to their hindered rotation around the amide bond.

The synthesis of the receptors 1 and 2 (scheme 1) started with the coupling of two equivalents of the mono-BOC-protected N-methyl-N,N-di(3-aminopropyl)amine 6 with the diacid chlorides of either fumaric acid (for 1) or maleic acid (for 2). The synthesis of the mono-BOC-protected species was straightforward and the excess of amine starting material was recovered from aqueous waste by drying and distillation. The resulting intermediates (9 and 10) were subsequently deprotected using gaseous HCl and isolated as hydrochloride salts after purification by preparative gel permeation chromatography (Sephadex G-10, eluent water). The degree of protonation was established by potentiometric titrations, vide infra.

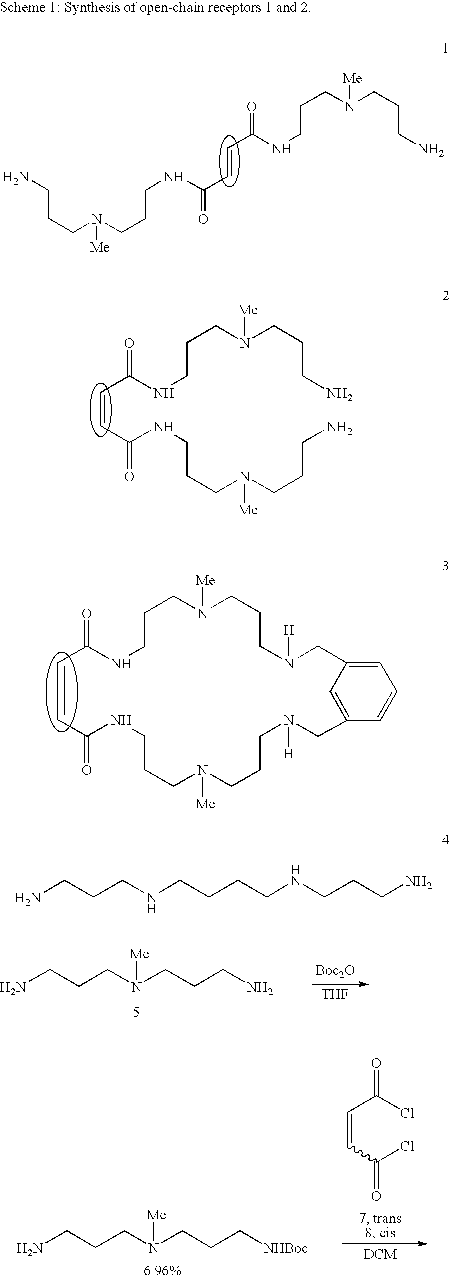

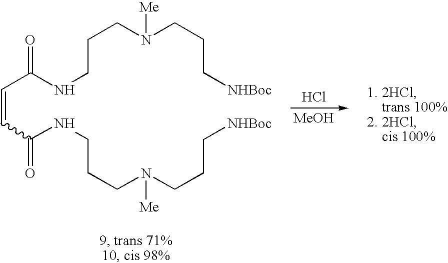

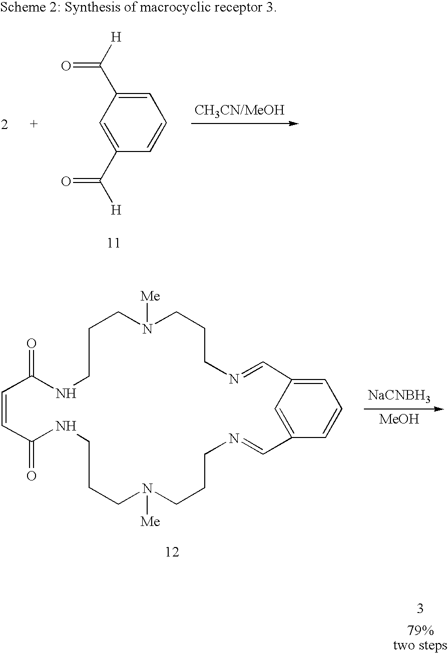

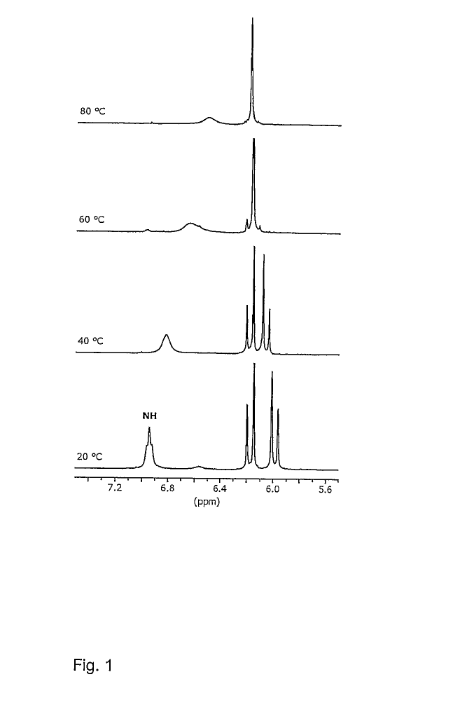

The difference between the two open-chain receptors is their configuration around the double bond. This difference leads to significant differences in the ¹H NMR spectra of this class of molecules. The Boc-protected trans compound 9 in [D6]DMSO displays the expected spectrum for a C₂ symmetric compound, but for the cis compound 10 in the same solvent the picture was different. Whereas for 9 only one singlet was observed for the double bond protons, for compound 10 a doublet of doublets arises in the spectrum (FIG. 1). Similarly, in the ¹³C spectrum, two signals are observed for the double bond carbons and there are also two discrete carbonyl signals. Apparently, compound 10 adopts a conformation, which removes the symmetry of the molecule.

When the temperature was increased from room temperature to 80° C., the doublets moved closer together and gradually changed into the expected singlet with coalescence occurring at 75° C. (FIG. 1). The peak at 6.92 ppm that belongs to one of the amide protons also changed from a relatively well-resolved triplet into a broad singlet that had shifted upfield. From the coalescence temperature the rotation barrier was determined to be 49±3 kJ mol⁻¹.

The shift and broadening of the NH signal indicates that a hydrogen bond may be involved in this conformational equilibrium, which slows down the rotation at room temperature. The most likely hydrogen bond is between the carbonyl of one amide and the NH of the other. Although this involves the formation of an energetically not very favourable seven membered ring, a similar structure has been proposed before for a related compound having two amino acids connected to maleic acid (S. Valenza et al., J. Org. Chem. 2000, 65, 4003-4008).

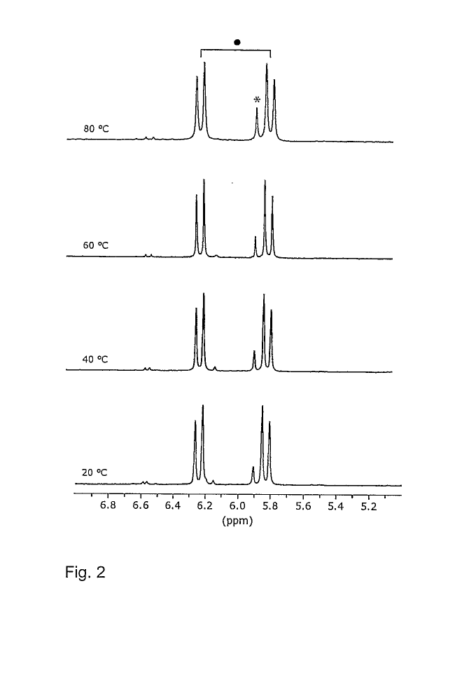

The same phenomenon of asymmetry is also observed for the final receptor 2 in D₂O, although in this case both conformations (the C₂ symmetric and the asymmetric conformer) are present at room temperature and are in slow exchange (FIG. 2). When the temperature is raised they remain in slow exchange, but their ratio changes in the favour of the symmetric conformation. This observation clearly indicates that the presence of amide groups on a conformationally restricted scaffold has pronounced structural effects on the receptor in aqueous conditions.

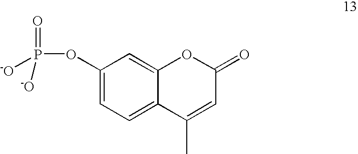

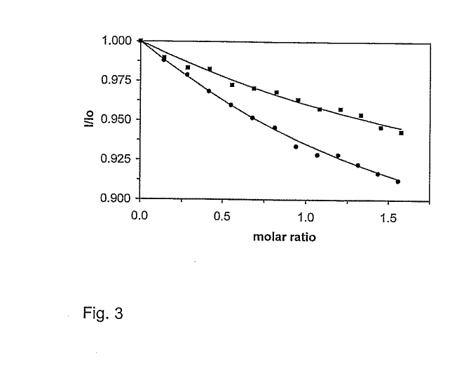

To rapidly assess the affinity of these receptors for phosphates we studied their binding of fluorescent phosphate derivative 13. Upon titration of our receptors to a solution of this probe in buffered water (0.1 M Tris, pH 7.0) the emission of compound 13 was quenched indicating binding (FIG. 3).

These data were subsequently fitted to a 1:1 binding model giving a binding constant (log K) of 5.15 for compound 2. Addition of compound 1 to 13 did not result in a significant quenching of the emission and its binding constant could therefore not be determined, indicating that no significant binding occurs. This is related to the trans configuration of the oligoamines around the conformationally restricted scaffold in compound 1 preventing the oligoamines from cooperating. The log K value for 2 is the highest that has been reported to date for a receptor of this kind binding a phosphate derivative in water under physiological conditions and indicates that this class of compounds are very promising anion receptors.

TABLE 1
Protonation constants (log K) of receptors 1-4.^{[a]}

| | 1 | 2 | 3 | 4 | 14 |
|---|---|---|---|---|---|
| H⁺ + L ⇄ HL⁺ | 10.45 | 10.04 | 10.32 | 10.64 | 9.76 |
| | (2) | (3) | (1) | (1) | (3) |
| 2H⁺ + L ⇄ H₂L²⁺ | 20.05 | 19.06 | 18.82 | 20.63 | 18.49 |
| | (2) | (3) | (1) | (1) | (3) |
| 3H⁺ + L ⇄ H₃L³⁺ | 28.42 | 26.52 | 25.14 | 29.50 | 25.67 |
| | (2) | (3) | (1) | (1) | (4) |
| 4H⁺ + L ⇄ H₄L⁴⁺ | 35.50 | 30.27 | 28.11 | 37.58 | 27.91 |
| | (2) | (4) | (2) | (1) | (9) |
| H⁺ + L ⇄ HL⁺ | 10.45 | 10.04 | 10.32 | 10.64 | 9.76 |
| H⁺ + HL⁺ ⇄ H₂L²⁺ | 9.60 | 9.02 | 8.50 | 9.99 | 8.73 |
| H⁺ + H₂L²⁺ ⇄ H₃L³⁺ | 8.37 | 7.46 | 6.32 | 8.87 | 7.18 |
| H⁺ + H₃L³⁺ ⇄ H₄L⁴⁺ | 7.08 | 3.75 | 2.97 | 8.08 | 2.24 |

^{[a]}Determined by potentiometry in 0.1 M KCl solution at 25° C.
Values in parentheses are standard deviations of the last decimal.

To study the binding in more detail we used potentiometry. To enable this, we first investigated the protonation constants of the receptors and the data is collected in Table 1. We calibrated our system with spermine (compound 4) and the data for this compound are also included in this table and were in accordance with literature values (Bazzicalupi et al, J. Am. Chem. Soc. 121: 6807-6815 (1999); De Stefano et al., J. Chem. Soc. Faraday Trans, 94:1091-1095 (1998); Bergeron et al., J. Med. Chem. 38:2278-2285 (1995)).

The number of atoms between two neighbouring amines in an oligoamine determines the difference in their pKₐ values. When there are two atoms separating the amines, the protonation of the second amine is strongly affected by the positive charge imposed by the protonation of the first amine, making it more difficult for the amine to accept a proton, i.e. lowering its pKₐ value. This effect is gradually reduced as the number of atoms separating the amines increases. For example, for 1,2-diaminoethane, the first protonation occurs at pKₐ=10.1 and the second at pKₐ=7.0, i.e a difference of 3.1 units, for 1,3-diaminopropane the first pKₐ=10.6 and the second pKₐ=8.6, a difference of 2 units, and for 1,4-diaminobutane the values are 10.8 and 9.4, a difference of only 1.4 units. (Bertsch et al., J. Phys. Chem. 62:444-446 (1958). To obtain a receptor with a high degree of protonation at neutral pH, therefore, it is important to consider the number of atoms between the amines.

The trans receptor 1 behaves like a typical open-chain polyamine with more than two atoms between the different amines such as spermine, whereby the consecutive protonation steps are only mildly affected by the presence of nearby protonated amines. The cis receptor, 2, on the other hand, although being an open-chain receptor, behaves more like a macrocycle. The third and especially the fourth protonation steps for 2 become significantly more difficult in comparison with 1. This is due to the cis configuration around the conformationally restricted scaffold, which concentrates the positively charged amines in a smaller area, much like a macrocycle does, i.e. the conformationally restricted scaffold turns this compound into a pseudo-macrocycle.

TABLE 2
Protonation and binding constants (Log K) of receptors 1-3 and 14.^{[a]}

| | 1 | 2 | 3 | 14 |
|---|---|---|---|---|
| 3H⁺ + L + A³⁻ ⇄ H₃LA | —^{[b]} | 34.94 (4) | 37.83 (4) | 34.34 (3) |
| 4H⁺ + L + A³⁻ ⇄ H₄LA⁺ | 42.87 | 43.27 (7) | 46.85 (4) | 42.67 (3) |
| | (4) | | | |
| 5H⁺ + L + A³⁻ ⇄ H₅LA²⁺ | 50.44 | 50.30 (7) | 53.76 (5) | 50.09 (3) |
| | (3) | | | |
| 6H⁺ + L + A³⁻ ⇄ H₆LA³⁺ | —^{[b]} | 56.33 (9) | 57.40 (5) | 56.47 (3) |
| H₂L²⁺ + HA²⁻ ⇄ H₃LA | —^{[b]} | 4.40 | 7.53 | 4.37 |
| H₃L³⁺ + HA²⁻ ⇄ H₄LA⁺ | 2.90 | 5.27 | 10.24 | 5.52 |
| H₄L⁴⁺ + HA²⁻ ⇄ H₅LA²⁺ | 3.39 | 8.55 | 14.17 | 10.70 |
| H₄L⁴⁺ + H₂A⁻ ⇄ H₆LA³⁺ | —^{[b]} | 7.67 | 10.90 | 10.17 |

^{[a]}Determined by potentiometry in 0.1 M KCl solution at 25° C. Values in parentheses are standard deviations of the last decimal. Protonation constants (log K) for phosphate were determined independently as 2.42, 6.91 and 11.48 respectively, in agreement with literature values.
^{[b]}Equilibrium does not occur significantly.

We then continued to investigate the binding of inorganic phosphate, again using potentiometry (Table 2). Potentiometry provides phosphate binding constants across the whole pH range, and thus gives a much more detailed picture of the binding event than the fluorimetric titrations. Compound 1 displays a moderate affinity towards phosphate, again typical for an open-chain receptor (log K=2.9-3.4). Receptor 2, with both arms directed to the same side of the molecule, displays much stronger binding (log K=4.4-8.6) because of its better organised binding site resulting from the cis arrangement around the conformational restricted scaffold. As expected, the data shows that receptors with higher degrees of protonation display the highest binding constants, demonstrating the importance of electrostatic interactions.

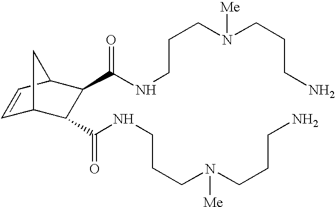

To further exemplify the general design principles of our receptor we synthesised compound 14 based on the rigid norbornene scaffold. The synthesis followed the same general route as for 1 and 2 and started by reacting two equivalents of compound 8 with the di-acid chloride derivative of 5-norbornene-2-endo,3-exo-dicarboxylic acid. Deprotection of the BOC-groups using gaseous HCl yielded the final receptor as its hydrochloride salt.

The protonation constants of 14 as measured by potentiometry (Table 1) are similar to 2 and this receptor also displays macrocycle-like behaviour, i.e. the third and fourth protonation steps become significantly more difficult than expected for a linear polyamine of this kind. In the case of receptor 14 the oligoamine arms are attached via amide bonds to the very conformationally restricted norbornene skeleton which allows virtually no conformational freedom. As a result, the fourth protonation constant in compound 14 is significantly lower compared to that of 2.

Receptor 14 displays the same high phosphate binding affinity as 2 and even shows slightly higher affinity in its fully protonated form (Table 2). This is a further demonstration that the more conformationally restricted the scaffold to which the oligoamine arms are attached the higher the phosphate binding affinity will be.

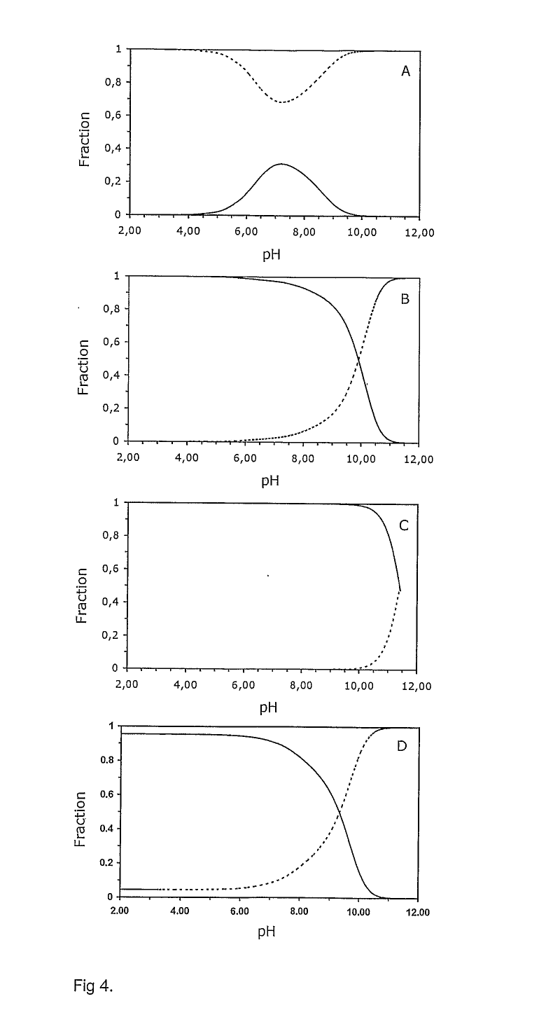

From the potentiometric data, speciation diagrams can be created for each of the receptors showing the fractions of the different phosphate bound species listed in Table 2 that are present at different pH values, compared to the fractions of free phosphates in solution. By adding up all the fractions of phosphate binding species and all the fractions of unbound phosphates, the graphs in FIG. 4 were created showing the overall efficiency of compounds 1, 2, and 14 to bind phosphate in solution over the whole pH range. This clearly shows the difference in affinity of these compounds. For example at pH 7.0, compound 1 binds only 31% of the phosphate in solution, whereas compounds 2 and 14 are able to bind as much as 95%.

These receptors show a higher affinity for inorganic phosphate than for phosphate 13. This reflects the fact that inorganic phosphate has an additional ionisable group and hence a higher charge density. It also has a significantly smaller size which will decrease steric repulsions.

The affinity of compounds 2 and 14 for inorganic phosphate is remarkably high. Indeed, it is higher than reported for macrocycles in the literature. The high affinity is due to the enhanced organisation of this receptor as a result of the conformational restraints introduced in the structure. These acyclic receptors therefore constitute a new class of easily synthesised, high affinity phosphate receptors.

We also synthesised macrocyclic compound 3 for comparison reasons by reacting compound 2 with isophthaldehyde under dilute conditions to promote macrocyclisation, rather than polymerisation (Scheme 2). The Schiff base intermediate 12 was not isolated, but immediately reduced to the corresponding amine 3 using sodium cyanoborohydride. Reduction with the more reactive sodium borohydride resulted in concomitant reduction of the double bond and was therefore avoided. The product was purified by preparative gel permeation chromatography and was isolated in its free base form.

The binding of compound 3 with phosphate 13 was also studied and the binding curve is shown in FIG. 3. Analysis of the binding yielded a logK value of 5.30, similar to the value of 5.15 observed for the binding of 13 to compound 2, demonstrating that the macrocylic compound also shows a high affinity for phosphates.

For more detail, the binding of ortho-phosphate was again studied using potentiometry. The pKa values of macrocyle 3 are listed in Table 1. A comparison of the data for 2 and 3 shows that the second, third and fourth pKₐ values for macrocycle 3 are all lower than the corresponding values for acyclic 2, even though the primary amines in 2 have been transformed into more basic secondary amines in 3. This is due to the macrocyclic effect, i.e. upon closing the ring, the ability of the amines to separate the positive charges is further restricted, and thus their consecutive pKₐ values are lowered.

The binding with inorganic phosphate to compound 3 was subsequently studied and the resulting binding constants are listed in Table 2. The macrocycle 3 has an even larger affinity for phosphate (log K=7.5-14.2) than its open-chain counter part 2 (Log K=4.4-8.6) due to a further increase of the conformational restraints of the receptor by the formation of the macrocyle. The data for 3 shows the same trend as for 2 that more charged receptors display higher binding. The values in Table 2 are the highest reported to date for the binding of ortho-phosphate in water by polyamine receptors.

When comparing the binding results of compound 2 and 3 for phosphate and for phosphate molecule 13 one can see that there is a clear difference in affinity for phosphate, with macrocyle 3 binding phosphate the strongest, but that the binding affinity of both receptors for compound 13 is virtually the same. This implies there is a clear advantage in using the open chain receptor when binding more complicated and potentially sterically hindered phosphate molecules, such as 13.

1. A ligand binding member comprising:
at least two oligoamines arranged for binding to a ligand; and
an organic template covalently linked to the at least two oligoamines such that movement of the oligoamines is conformationally restricted wherein the oligoamines are the same or different and are independently straight chained, cyclic or branched.

2. A ligand binding member as claimed in claim 1 which consists of two oligoamines.

3. A ligand binding member as claimed in claim 1 wherein the oligoamines are the same.

4. A ligand binding member as claimed in claim 3 wherein the oligoamines are located cis or trans with respect to each other.

5. A ligand binding member as claimed in claim 1 wherein the at least two oligoamines are independently acyclic oligoamines.

6. A ligand binding member as claimed in claim 1 wherein the template is an unsaturated hydrocarbon, a cyclic aliphatic hydrocarbon or a cyclic aromatic hydrocarbon.

7. A ligand binding member as claimed in claim 6 wherein the unsaturated hydrocarbon is an alkene.

8. A ligand binding member as claimed in claim 6 wherein the unsaturated hydrocarbon is an alkyne.

9. A ligand binding member as claimed in claim 6 wherein the cyclic aromatic hydrocarbon is a monocyclic or polycyclic aromatic hydrocarbon.

10. A ligand binding member as claimed in claim 9 wherein the polycyclic aromatic hydrocarbon comprises two, three or four hydrocarbon rings.

11. A ligand binding member as claimed in claim 9 wherein the polycyclic aromatic hydrocarbon is selected from the group consisting of naphthalene, indene, pentalene, azulene, heptalene, biphenylene, indacene, fluorene, phenalene, phenanthrene, anthracene, pyrene, chrysene, naphthacene, acephananthrylene, aceanthrylene, triphenylene and fluoroanthene.

12. A ligand binding member as claimed in claim 6 wherein the cyclic aliphatic hydrocarbon is a monocyclic or polycyclic aliphatic hydrocarbon.

13. A ligand binding member as claimed in claim 12 wherein the monocyclic aliphatic hydrocarbon comprises between 3 and 10 carbon atoms.

14. A ligand binding member as claimed in claim 12 wherein the polycyclic aliphatic hydrocarbon comprises two, three or four hydrocarbon rings.

15. A ligand binding member as claimed in claim 12 wherein the polycyclic aliphatic hydrocarbon is a bicyclic ring system.

16. A ligand binding member as claimed in claim 6 wherein the template is a heterocyclic hydrocarbon.

17. A ligand binding member as claimed in claim 1 wherein the template is a sugar or glycoside.

18. A ligand binding member as claimed in claim 17 wherein the sugar is a pentose or hexose sugar.

19. A ligand binding member as claimed in claim 1 which further comprises one or more amide groups.

20. A ligand binding member as claimed in claim 19 wherein the at least two oligoamines are covalently linked to the template.

21. A ligand binding member as claimed in claim 20 wherein the at least two oligoamines are covalently linked to the template via one or more amide bonds.

22. A ligand binding member as claimed in claim 1 wherein the at least two oligoamines are made up of primary, secondary or tertiary amines, quaternary ammonium salts or a combination thereof.

23. A ligand binding member as claimed in claim 22 wherein the at least two oligoamines comprise a primary amine group and a tertiary amine group.

24. A ligand binding member as claimed in claim 1 wherein the oligoamines contain between 2 and 4 protonatable amines.

25. A ligand binding member as claimed in claim 24 wherein the oligoamines contain between 3 and 4 protonatable amines.

26. A ligand binding member as claimed in claim 1 wherein the amine groups of the oligoamines are separated by at least two atoms.

27. A ligand binding member as claimed in claim 26 wherein the oligoamines are separated by 3 or 4 atoms.

28. A ligand binding member as claimed in claim 26 wherein the atoms are carbon, oxygen or sulphur.

29. A ligand binding member as claimed in claim 1 wherein the ligand binding member is a receptor.

30. A ligand binding member as claimed in claim 29 wherein the receptor is an acyclic receptor.

31. A ligand binding member as claimed in claim 1 which is synthetic.

32. A ligand binding member as claimed in claim 1 wherein the ligand is a phosphate molecule or ion.

33. A ligand binding member as claimed in claim 30 wherein the phosphate molecule is an inorganic phosphate molecule or organic phosphate molecule.

34. A ligand binding member as claimed in claim 1 wherein the ligand is a phosphate containing biological molecule.

35. A ligand binding member as claimed in claim 34 wherein the biological molecule is a protein, lipid, carbohydrate or nucleic acid.

36. A ligand binding member as claimed in claim 1 wherein the ligand binding member is a phosphatase inhibitor.

37. A ligand binding member as claimed in claim 1 for use as a medicament.

38. A ligand binding member as claimed in claim 1 wherein the ligand binding member is immobilised on a solid support.

39. A ligand binding member as claimed in claim 38 wherein the solid support is a polymer.

40. A ligand binding member as claimed in claim 39 wherein the polymer is selected from the group consisting of vinyl type polymers, acrylic polymers, allyl polymers and polyethyleneoxides.

41. A ligand binding member as claimed in claim 39 wherein the polymer is selected from the group consisting of agarose, sepharose, dextran, cellulose, xylan, lignin, nylon, DNA, RNA, polyester, polyamide, polycarbonate, and derivatives thereof.

42. A ligand binding member as claimed in claim 38 wherein the support is in the form of polymer beads, glass beads, or silica beads.

43. A ligand binding member as claimed in claim 38 wherein the support is a microtitre plate, glass slide, a gel, fibres, filaments, membrane, dendrites, resins, micelles, nanoparticles or phase interfaces.

44. A ligand binding member as claimed in claim 38 wherein the support is part of a column, filtration device, mesh or gel.

45. A ligand binding member as claimed in claim 1 which comprises a detectable label.

46. A ligand binding member as claimed in claim 45 wherein the label is an enzyme, a fluorescent label or a radioisotope.

47. A filter comprising a ligand binding member as claimed in claim 1.

48. A water purification system comprising a filter as claimed in claim 47.

49. A renal dialysis system comprising a filter as claimed in claim 47.

50. The use of a ligand binding member as claimed in claim 1 in the removal of phosphate molecules, phosphate ions or phosphate containing molecules from a liquid.

51. The use as claimed in claim 50 wherein the liquid is water or effluent.

52. The use as claimed in claim 50 wherein the liquid is blood.

53. The use of a ligand binding member as claimed in claim 1 in the selective screening of phosphate molecules or phosphate containing molecules.

54. The use as claimed in claim 53 wherein the phosphate molecule is an inorganic phosphate.

55. The use as claimed in claim 53 wherein the ligand binding member is cyclic.

56. A process of separating a phosphate molecule or ion, or phosphate containing molecule, from a liquid, the process comprising:
contacting a ligand binding member as claimed in claim 1 with a liquid at pH values between about 5 and 9; and
optionally eluting any phosphate molecule, ion or phosphate containing molecule which bound to the ligand binding member.

57. The use of a ligand binding member as claimed in claim 1 in the inhibition of phosphatase activity.

58. The use of a ligand binding member as claimed in claim 1 in the manufacture of a medicament for the treatment or prevention of renal failure.

59. A method of treating renal failure by prophylaxis or therapy, comprising administration to a subject of a therapeutically effective amount of a ligand binding member as claimed in claim 1.
